(19) United States
(12) Patent Application Publication
(10) Pub. No.: US 2018/0360361 A1
(43) Pub. Date: Dec. 20, 2018

(54) CORD BLOOD COLLECTION SYSTEM AND METHOD OF OPERATION

(71) Applicants: Zack Cleary, Rockville, MD (US); Luis Montes, (US); Saleh Alamri, (US); UNIVERSITY OF HARTFORD, West Hartford, CT (US)

(72) Inventors: Andrea Kwaczala, Cheshire, CT (US); Zack Cleary, Rockville, MD (US); Luis Montes, Guaynabo (PR); Saleh Alamri, Ar-Riyadh (SA)

(21) Appl. No.: 16/008,732

(22) Filed: Jun. 14, 2018

Related U.S. Application Data
(60) Provisional application No. 62/520,729, filed on Jun. 16, 2017.

(51) Int. Cl. A61B 5/15

(52) U.S. Cl. CPC A61B 5/150038; A61B 5/150045; A61M 1/1039; A61B 5/150366; A61M 2210/1466

(57) ABSTRACT

A cord blood collection system is provided. The system includes a housing having an upper portion and a lower portion, the upper portion and lower portion defining a hollow interior. A peristaltic pump is disposed within the lower portion, the peristaltic pump having an input portion and an output portion. A reel member is disposed in the upper portion. A tubing member is movably coupled to the reel member, the tubing member being operably coupled to the peristaltic pump, the first end being configured to couple with a container, the second end being configured to couple with a needle.

### CROSS REFERENCE TO RELATED APPLICATIONS

This application claims the benefit of U.S. Provisional Application Ser. No. 62/520,729, filed Jun. 16, 2017, the entire disclosure of which is incorporated herein by reference.

### BACKGROUND

The subject matter disclosed herein relates to a system for collecting blood from an umbilical cord and the placenta and in particular to a system that is low in cost and rapidly collects cord blood.

The blood from a human umbilical cord, sometime referred to as “cord blood” is rich source of stem cells that have many therapeutic benefits. Cord blood has been used as a treatment for more than 70 different diseases, including cancers, blood disorders, and immune deficiencies. Further, there is a high probability that many therapeutic treatments that utilize cord blood have yet to be discovered. Despite these potential health benefits, only ˜5% of all parents chose to collect and store their newborn baby's cord blood. This is due to lack of availability to collection tools in the hospital, the time associated with collection after child birth and the expenses associated with long term storage.

Typically, the collection of cord blood is initiated after the umbilical cord stops pulsating and the cord is clamped and cut. There are two common collection procedures, known as the “bag method” and the “syringe method.” With the bag method, the placenta and cord are elevated, using the force of gravity to drain the blood from the placenta into the bag, but this method can take nearly 40 minutes to collect an adequate sample, delaying delivery of the placenta. With the syringe method, the procedure is similar to that of a blood draw, an empty syringe is inserted into the umbilical cord artery and is used to draw blood from within the vasculature of remaining cord, but only small volumes of blood can be collected with this method.

Accordingly, while existing cord blood collection techniques are suitable for their intended purpose the need for improvement remains, particularly in providing a system that rapidly collects the cord blood in a cost efficient manner.

### BRIEF DESCRIPTION

According to one aspect of the disclosure a cord blood collection system is provided. The system includes a housing having an upper portion and a lower portion, the upper portion and lower portion defining a hollow interior. A peristaltic pump is disposed within the lower portion, the peristaltic pump having an input portion and an output portion. A reel member is disposed in the upper portion. A tubing member is movably coupled to the reel member, the tubing member being operably coupled to the peristaltic pump, the first end being configured to couple with a container, the second end being configured to couple with a needle.

According to another aspect of the disclosure a method of collecting cord blood is provided. The method includes coupling a tubing member to a reel member. Coupling the tubing member to a peristaltic pump. Coupling a first end of the tubing member to a container. Coupling the second end of the tubing member to a needle. The needle is inserted into a vasculature of an umbilical cord immediately after the delivery of a newborn infant. The peristaltic pump is activated and blood is transferred from the vasculature to the container in response to the activation of the peristaltic pump.

These and other advantages and features will become more apparent from the following description taken in conjunction with the drawings.

### BRIEF DESCRIPTION OF DRAWINGS

The subject matter, which is regarded as the disclosure, is particularly pointed out and distinctly identified in the claims at the conclusion of the specification. The foregoing and other features, and advantages of the disclosure are apparent from the following detailed description taken in conjunction with the accompanying drawings in which:

FIG. 1 is a perspective view of the cord blood collection system in accordance with an embodiment;

FIG. 2 is a disassembled view of the cord blood collection system of FIG. 1;

FIG. 3 is a schematic view of a peristaltic pump for use in the cord blood collection system of FIG. 1;

FIG. 4 is a top view of the housing of the cord blood collection system of FIG. 1; and

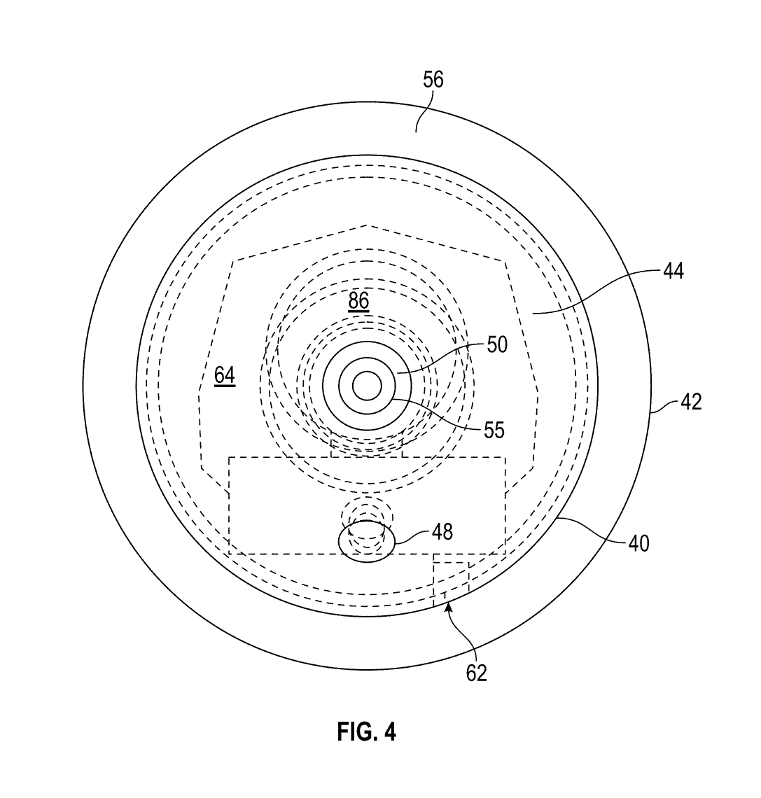

FIG. 5 is a side view of the housing of FIG. 4.

The detailed description explains embodiments of the disclosure, together with advantages and features, by way of example with reference to the drawings.

### DETAILED DESCRIPTION

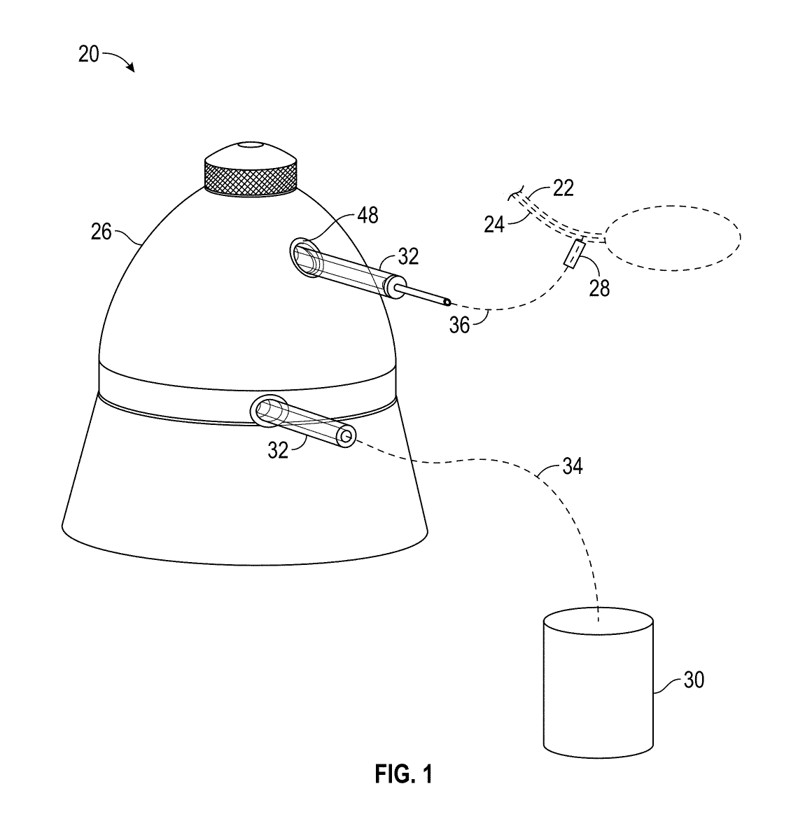

Embodiments of the invention provide advantages in the collection of cord blood from an umbilical cord and placenta in a rapid and cost effective manner. Referring now to FIG. 1, an embodiment of a cord blood collection system 20 is shown for acquiring blood from the vasculature 22 of an umbilical cord 24. The system 20 includes a housing assembly 26 that is fluidly coupled between a storage container 30 and a needle 28 that pierces the vasculature 22. In an embodiment, the housing 26 includes a retractable tube 32 having a first portion 34 that couples to the storage container 30 and a second portion 36 that couples to the needle 28. As will be discussed in more detail herein, the tube 32 may be selectively retracted and stored in the housing assembly 26.

It should be appreciated that while embodiments herein refer to the storage container 30 as a bottle, vial or bag, the claims should not be so limited. In other embodiments, the storage container 30 may be any vessel suitable for receiving and holding the cord blood in accordance with standard bodily fluid handling procedures.

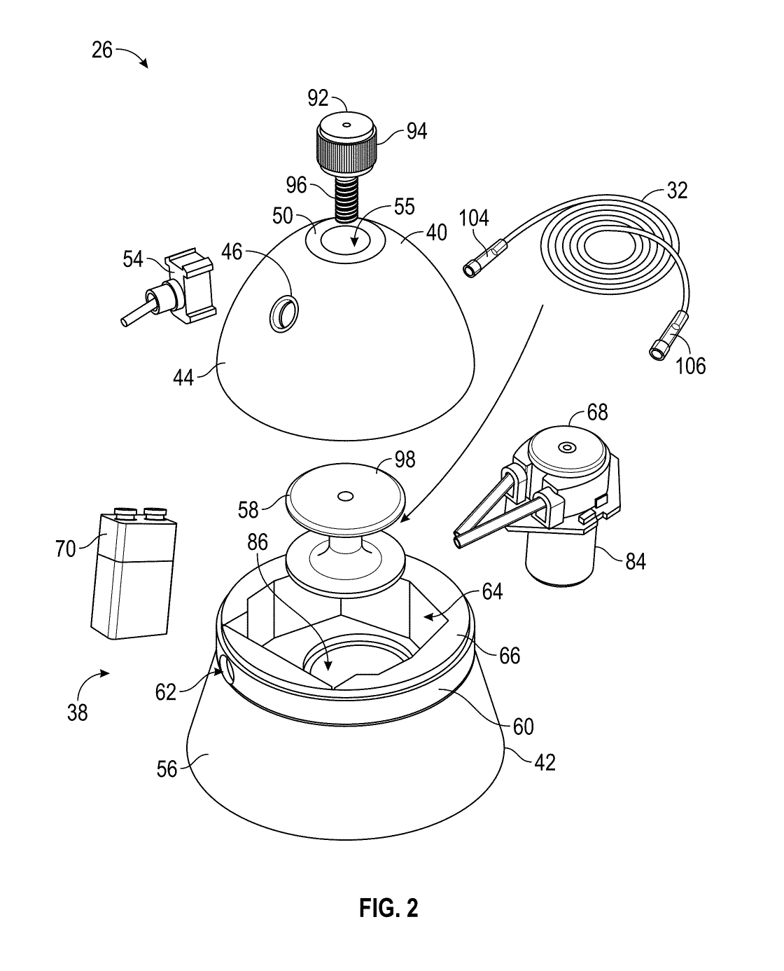

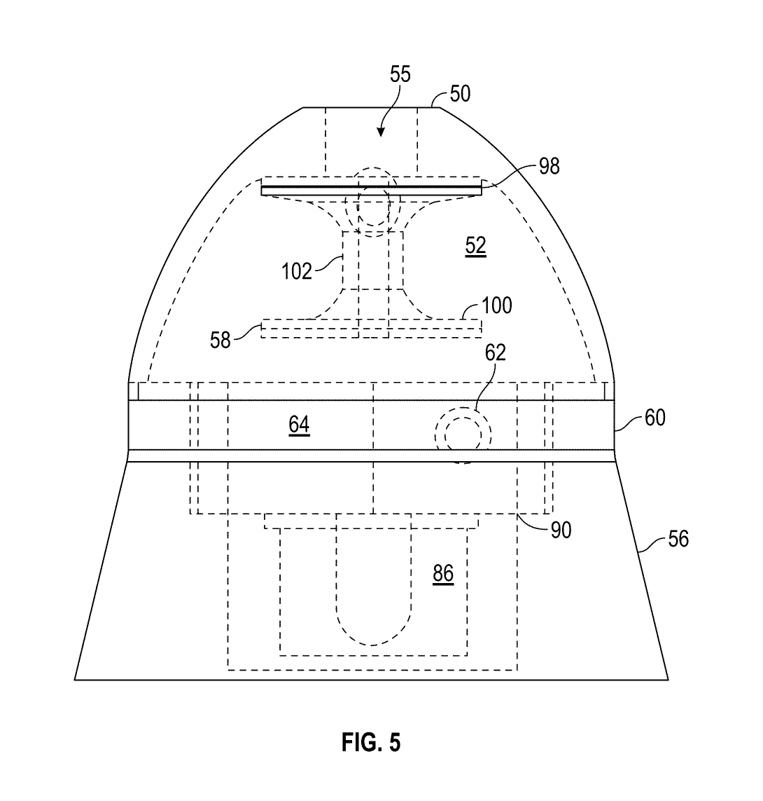

Referring now to FIG. 2, an embodiment is shown of the housing assembly 26. The housing assembly 26 includes a housing 38 having an upper portion 40 and a lower portion 42. The housing portions 38, 40 are removably coupled to each other. In an embodiment, the coupling is in the form of a lap joint. In an embodiment, the upper portion 40 includes a curved wall 44 and a substantially planar top surface 50. A hole 55 extends through the surface 50 into an upper housing space 52 (FIG. 5) defined by the wall 44. In an embodiment, the wall 44 has an ovoid shape. As will be discussed in more detail herein, the upper housing space 52 is sized to receive a retractable tubing reel member 58. The wall 44 further includes a first opening 46 and a second opening 48 (FIG. 1). The first opening 46 receives a switch 54 that allows the operator to activate the motor in the system 20 to pump cord blood from the vasculature 22 into the container 30.

In an embodiment, the lower portion 42 includes a conically shaped outer surface 56 with a cylindrical portion 60 extending therefrom. An opening 62 extends through the cylindrical portion 60 into an opening 64 that extends from a top surface 66. The opening 64 is sized to receive a peristaltic pump 68 and an energy source, such as a battery 70. The switch 54 is electrically connected between the battery 70 and the peristaltic pump 68 to allow the operator to control the operation of the peristaltic pump 68.

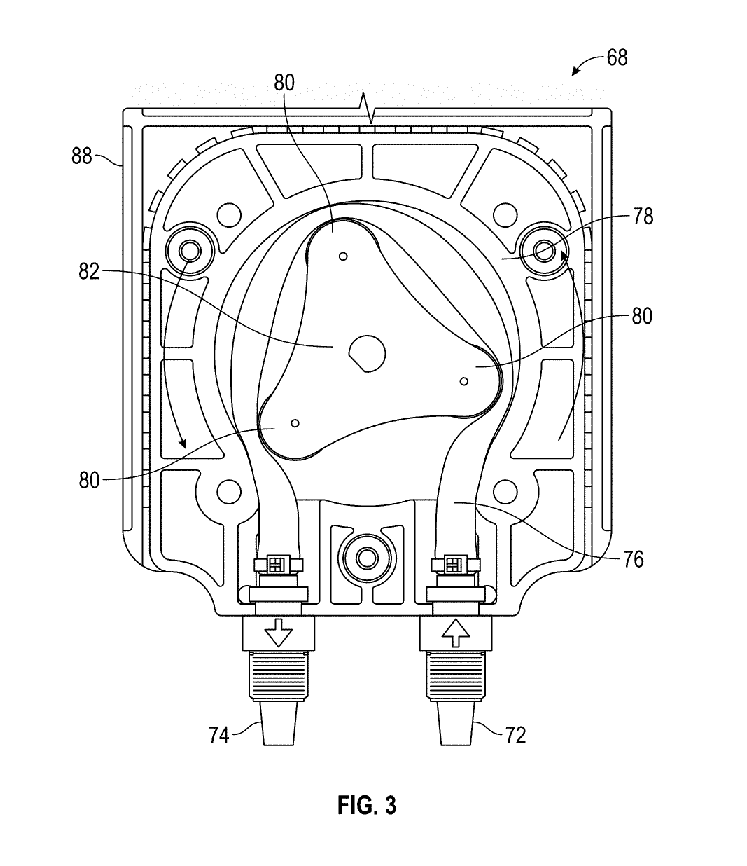

In an embodiment shown in FIG. 3, the peristaltic pump 68 includes an input port 72 and an output port 74. The ports 72, 74 are connected by a flexible tube 76 that is disposed between a housing wall 78 and the lobes 80 of a rotor 82. The rotor 82 is rotated by a motor 84 (FIG. 2). When the motor 84 is energized, the lobes 80 rotate to compress the tube 76. This compression forces the fluid within the tube 76 to be moved from the inlet 72 to the outlet 74. This type of pump is sometimes referred to as a positive displacement pump. It should be appreciated that peristaltic pumps provide advantages in that they allow the movement of blood, such as cord blood for example without causing significant hemolysis which is desirable for this application.

In an embodiment, the motor portion 84 of the peristaltic pump 68 is positioned within a recession 86 of the system to allow the pump casing 88 to sit on a surface 90 within the opening 64. When positioned in this manner, the output port 74 may be approximately aligned with the opening 62. It be appreciated that the positioning of the motor 84 within the hole 86 along with the shape of the housing lower portion 42 also provides a center of gravity to improve stability of the housing assembly 26. In an embodiment, the input port 72 is fluidly coupled to the tubing second portion 36 and the output port 74 is fluidly coupled to the tubing first portion 34. In an embodiment, the flexible tube 76 and ports 72, 74 are replaceable and disposable to allow the pump 68 to be reused without having to use extensive sterilization or cleaning procedures between patients. In still another embodiment, the casement portion 88 (including the rotor 82, tube 76 and ports 72, 74) is removable and disposable allowing the motor 84 to be reused.

In an embodiment, the tubing second portion 36 may be extended from and retracted to the housing 38, this allows accessibility to the device from the table or the floor below to connect to the patient. In this embodiment, the reel member 58 is rotatably coupled to the housing upper portion 44 by a turning member 92. The turning member 92 may include a knob 94 and a threaded portion 96. A bottom surface of the knob 94 rests on the top surface 50 and the threaded portion 96 extends through the hole 54 and couples to a first end 98 of the reel member 58. In an embodiment, the reel member 58 includes a pair of opposing ends 98, 100 with a stem 102 (FIG. 5) disposed there between. The stem 102 being smaller than the ends 98, 100 defines a space that allows the tubing 32 to be wrapped around the stem 102 within the space. In the exemplary embodiment, there is sufficient space for the housing upper portion 44 to contain 3 feet (0.91 meters) of standard medical grade tubing on the reel member 58. To extend the tubing 32, the operator pulls on a first end 104 of the tubing 32, causing the reel member 58 and the turning member 92 to rotate as the tubing 32 is extended. Once the desired amount of tubing 32 has been pulled from the housing 38, the operator may couple a second end 106 to the input 72 of the peristaltic pump 68. A second tube 32 is coupled between the output port 74 and the storage container 30.

Once the needle 28 is coupled to the first end 104, the operator may puncture the vasculature 22. By actuating the switch 54, the motor 84 is energized by the battery 70 causing the rotor 82 and lobes 80 to rotate and draw cord blood from the vasculature 22. The cord blood is drawn through the tubing 32 and deposited within the storage container 30. When the collection is completed, the operator actuates the switch 54 to de-energize the motor 84. To retract the tubing second portion 36, the operator rotates the knob 94 to rotate the reel member 58. The rotation of the reel member 58 cause the tubing 32 to be moved through the hole 46 and into the upper housing space 52.

It should be appreciated that while the switch 54 is illustrated as a toggle type switch, this is for exemplary purposes and the claims should not be so limited. In other embodiments, the switch 54 may include a rotary switch potentiometer that allows modulation of the voltage from the energy source to control pump speed.

It should also be appreciated that materials used in construction of the reusable components of the housing assembly 26, such as the housing 38, the reel member 58 and the turning member 92 may be selected to be sterilized using one or more common medical sterilization techniques, such as but not limited to steam sterilization, dry heat sterilization (autoclaving), chemical sterilization (ethylene oxide, sporicidal chemicals) and irradiation for example.

The term “about” is intended to include the degree of error associated with measurement of the particular quantity based upon the equipment available at the time of filing the application. For example, “about” can include a range of ±8% or 5%, or 2% of a given value.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the disclosure is provided in detail in connection with only a limited number of embodiments, it should be readily understood that the disclosure is not limited to such disclosed embodiments. Rather, the disclosure can be modified to incorporate any number of variations, alterations, substitutions or equivalent arrangements not heretofore described, but which are commensurate with the spirit and scope of the disclosure. Additionally, while various embodiments of the disclosure have been described, it is to be understood that the exemplary embodiment(s) may include only some of the described exemplary aspects. Accordingly, the disclosure is not to be seen as limited by the foregoing description, but is only limited by the scope of the appended claims.

What is claimed is:

1. A cord blood collection system comprising:
a housing having an upper portion and a lower portion, the upper portion and lower portion defining a hollow interior;
a peristaltic pump disposed within the lower portion, the peristaltic pump having an input portion and an output portion;
a reel member disposed in the upper portion; and
a tubing member movably coupled to the reel member, the tubing member being operably coupled to the peristaltic pump, the first end being configured to couple with a container, the second end being configured to couple with a needle.

2. The system of claim 1, further comprising:
a switch electrically coupled to the peristaltic pump; and
an energy source electrically coupled to the switch.

3. The system of claim 2, wherein the energy source is a battery disposed within the hollow interior.

4. The system of claim 1, further comprising the container having a port configured to couple with the first end.

5. The system of claim 4, wherein the container is one of a collection bottle, a collection vial or a collection bag.

6. The system of claim 1, wherein the upper portion includes:
a curved wall defining an upper housing space, the reel member being disposed within the upper housing space; and
a first opening extending through into the upper housing space.

7. The system of claim 6, further comprising a turning member that at least partially extends through the opening and couples to the reel member, the turning member being rotationally coupled to the upper portion when the turning member is coupled to the reel member.

8. The system of claim 6, wherein the curved wall has an ovoid shape.

9. The system of claim 8, wherein the lower portion includes a conical surface and a substantially planar bottom surface.

10. The system of claim 9, wherein the lower portion includes a second opening extending from a top surface opposite the bottom surface.

11. The system of claim 10, wherein the second opening is sized to receive the peristaltic pump.

12. The system of claim 11, wherein:
the upper portion includes a first hole extending through the curved wall, the first hole sized to allow the tubing member to pass therethrough; and
the lower portion includes a second hole extending through an outer surface into the second opening, the second hole being sized to allow the tubing member to pass therethrough.

13. A method of collecting cord blood, the method comprising:
coupling a tubing member to a reel member;
coupling the tubing member to a peristaltic pump;
coupling a first end of the tubing member to a container;
coupling the second end of the tubing member to a needle;
inserting the needle into a vasculature of an umbilical cord immediately after the natural delivery of a newborn infant;
activating the peristaltic pump; and
transferring blood from the vasculature to the container in response to the activation of the peristaltic pump.

14. The method of claim 13, further comprising:
rotatably coupling the reel member to an upper portion of a housing prior to coupling the tubing member to the reel member;
disposing the peristaltic pump within a lower portion of the housing.

15. The method of claim 14, further comprising disposing an energy source within the lower portion and selectively coupling the energy source to the peristaltic pump.

16. The method of claim 15, wherein the upper portion includes a curved wall defining an upper housing space, the upper housing space being sized to receive the reel member.

17. The method of claim 16, further comprising:
removing the needle from the vasculature; and
rotating the reel member to dispose at least a first portion of the tubing member between the reel member and the second end within the upper housing space.

18. The method of claim 16, wherein the lower portion includes a first opening sized to receive the peristaltic pump.

19. The method of claim 18, wherein the first portion extends through a first hole that extends through the curved wall.

20. The method of claim 19, wherein a second portion of the tubing member extends through a second opening, the second opening extending between an outer surface of the lower portion and the first opening.
